(19) United States
(12) Patent Application Publication
(10) Pub. No.: US 2015/0117738 A1
(43) Pub. Date: Apr. 30, 2015

(54) CT SYSTEM WITH COMPUTER UNIT AND METHOD FOR RECONSTRUCTING AND DIAGNOSING VISUAL CT RENDERINGS

(71) Applicant: Siemens Aktiengesellschaft, Munich (DE)

(72) Inventors: Christoph BRAUN, Rosenheim (DE); Robert LAPP, Nuernberg (DE); Michael SCHEUERING, Nuernberg (DE); Grzegorz SOZA, Heroldsberg (DE); Johann UEBLER, Nuernberg (DE)

(21) Appl. No.: 14/494,755

(22) Filed: Sep. 24, 2014

(30) Foreign Application Priority Data
Oct. 24, 2013 (DE) 102013221603.3

(51) Int. Cl. G06T 7/00; A61B 6/00; A61B 6/03

(52) U.S. Cl. CPC G06T 7/0012; A61B 6/03; A61B 6/5205
USPC 382/131

(57) ABSTRACT

An embodiment of a CT system includes a computer unit and at least one memory device for storing program code and projection data records, established during a CT scan and usable for reconstructing CT renderings. The computer unit is connected in a networked system with at least one external diagnosis computer including a memory with program code for diagnosing reconstructed visual CT renderings. A method for reconstructing and diagnosing visual CT renderings is also disclosed. In an embodiment, a request for reconstructing a CT data record with a projection data record stored in the computer unit is received, sent from a diagnosis computer in a diagnosis station, by the computer unit of the CT system via the communications network; the computer unit recognizes this request; the computer unit automatically executes the requested reconstruction and the reconstructed CT rendering is transmitted to the requesting diagnosis computer via the communications network.

### PRIORITY STATEMENT

The present application hereby claims priority under 35 U.S.C. §119 to German patent application number DE 102013221603.3 filed Oct. 24, 2013, the entire contents of which are hereby incorporated herein by reference.

### FIELD

At least one embodiment of the invention generally relates to a CT system, comprising a computer unit with at least one memory device for storing program code, which is executed during the operation, and for storing projection data records, which were established during a CT scan and which can be used for reconstructing CT renderings, wherein the computer unit is connected in a networked system with at least one external diagnosis computer for diagnosing reconstructed visual CT renderings.

Moreover, at least one embodiment of the invention generally relates to a method for reconstructing and diagnosing visual CT renderings, wherein at least one CT system comprising a computer unit with a memory and at least one diagnosis station spatially separate from the CT system and comprising a diagnosis computer with a memory are connected to one another via a communications network, wherein program code for carrying out the method steps described below is stored in the memories of the computer unit and of the diagnosis computer.

### BACKGROUND

The prior art has disclosed such CT systems, which are in a networked system with diagnosis stations and transmit CT renderings to the diagnosis stations via the networked system. If additional CT renderings with other reconstruction parameters are desired for an improved diagnosis, this is communicated to the operating staff at the CT system, who then initiate the corresponding reconstruction with the desired reconstruction boundary conditions and ensure that the CT renderings reconstructed thus are forwarded to the requesting diagnosis station.

### SUMMARY

The inventors have recognized that a problem is that the reconstruction of new CT renderings is only possible if respective operating staff are available at the CT system. However, since the work hours on the CT system and the work hours at the diagnosis stations are not necessarily in sync, there may be unwanted delays in the reconstruction of new CT renderings and hence the diagnosis itself may be unnecessarily delayed.

At least one embodiment of the present invention is directed to a CT system in a networked system with diagnosis stations, and a method for diagnosis, such that complementary or new CT renderings, which are reconstructed under arbitrary reconstruction boundary conditions, can be called at the diagnosis station without operating staff needing to be present at the CT system.

Advantageous developments of the invention are the subject matter of the dependent claims.

The inventors have discovered that it is also possible to obtain complementary CT renderings with the desired reconstruction boundary conditions at a diagnosis station outside of the work hours on a CT system if the CT system is configured in such a way that reconstruction requests can be transmitted directly via a networked system to the computer unit of the CT system. Here, the computer unit of the CT system should, where necessary, also be responsive in a stand-by mode such that a trigger signal coming in via a network connection can be identified at all times so that a request for a reconstruction under predetermined boundary conditions and using available projection data records is carried out and the result thereof is transmitted to the requesting diagnosis station. As long as the computational resources of the computer unit are not overwhelmed in relation to the additionally requested reconstruction, the operating state of the scanning system with gantry, x-ray tubes and detector is irrelevant in relation to the stand-by mode. However, a request list, which is worked through as soon as appropriate computational resources are available, could be formed in such a case.

Accordingly, in at least one embodiment, the inventors propose a CT system comprising a computer unit with at least one memory device for storing program code, which is executed during the operation, and for storing projection data records, which were established during a CT scan and which can be used for reconstructing CT renderings, wherein the computer unit is connected in a networked system with at least one external diagnosis computer comprising a memory with program code for diagnosing reconstructed visual CT renderings. According to at least one embodiment of the invention, a program code is to be available in the memory of the computer unit, which program code is executed both during operation and in the “stand-by” state, wherein a new reconstruction of a CT rendering from a projection data record previously stored in the memory is executed or tasked, controlled by this program code and an external trigger signal emitted by a diagnosis computer present in the networked system, and the newly reconstructed CT rendering is sent to this diagnosis computer.

In accordance with the CT system according to at least one embodiment of the invention, the inventors also propose a method for reconstructing and diagnosing visual CT renderings, wherein at least one CT system comprising a computer unit with a memory and at least one diagnosis station spatially separate from the CT system and comprising a diagnosis computer with a memory are connected to one another via a communications network, wherein program code for carrying out the method steps described below is stored in the memories of the computer unit and of the diagnosis computer. Accordingly, the following is intended:

a request for reconstructing a CT data record with a projection data record stored in the computer unit is to be sent from a diagnosis computer in a diagnosis station to the computer unit of the CT system via the communications network,

the computer unit is to recognize this request,

the computer unit is to automatically execute the requested reconstruction, that is to say, with no additional action by operating staff, and

the reconstructed CT rendering is to be transmitted to the requesting diagnosis computer via the communications network.

Moreover, according to at least one embodiment of the invention, a request profile, which defines a diagnostic object, can also be sent from the diagnosis computer to the computer unit and the computer unit extracts the reconstruction parameters from a predetermined table, in which reconstruction parameters which have an ideal configuration for the diagnostic question are stored for each request profile, on the basis of the request profile.

### BRIEF DESCRIPTION OF THE DRAWINGS

Below, embodiments of the invention will be described in more detail with the aid of the figures, with only the features necessary for understanding the invention being depicted. The following reference signs are used: 1: CT system; 2: gantry; 3: patient; 4: computer unit; 5, 6: diagnosis computer; 11, 12: diagnosis station; E: communications network; P1-Pk: projection data records; Prg1-Prgn: program code in the computer unit; Prg1-Prgm: program code in the diagnosis computer; S1-S5: method steps.

In detail:

FIG. 1 shows a networked system comprising a CT system and two diagnosis stations;

FIG. 2 shows a flowchart of a method process according to an embodiment of the invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

Accordingly, in at least one embodiment, the inventors propose a CT system comprising a computer unit with at least one memory device for storing program code, which is executed during the operation, and for storing projection data records, which were established during a CT scan and which can be used for reconstructing CT renderings, wherein the computer unit is connected in a networked system with at least one external diagnosis computer comprising a memory with program code for diagnosing reconstructed visual CT renderings. According to at least one embodiment of the invention, a program code is to be available in the memory of the computer unit, which program code is executed both during operation and in the “stand-by” state, wherein a new reconstruction of a CT rendering from a projection data record previously stored in the memory is executed or tasked, controlled by this program code and an external trigger signal emitted by a diagnosis computer present in the networked system, and the newly reconstructed CT rendering is sent to this diagnosis computer.

Here, at least one CT slice image and/or a 3D rendering is reconstructed as CT rendering, wherein the specific type of reconstruction is also transmitted with the request by way of appropriate reconstruction parameters.

Furthermore, the suggestion is made that the program code available in the memory of the computer unit brings about the reception of at least one parameter which permits a unique identification of the projection data record to be used for the reconstruction.

In order to enable the CT system to obtain ideal prescriptions from the requesting diagnosis computer for reconstruction purposes, the CT system should, at least in respect of the programming thereof, be configured in such a way that the program code available in the memory of the computer unit brings about the reception of at least one reconstruction parameter, which is used for the reconstruction.

In principle, all reconstruction parameters required for the reconstruction can be transmitted in this manner. However, if only some of the required reconstruction parameters are transmitted, the program code available in the memory of the computer unit can be configured in such a way that non-received reconstruction parameters are automatically replaced by standard values.

Furthermore, the suggestion is made that the CT system is configured in such a way that, after receiving a trigger signal, the program code available in the memory of the computer unit puts the computer unit of the CT system out of the “stand-by” state and into an operating state without activating the gantry, x-ray tubes and detector. As a result, the CT system can be shut down with the exception of the computer unit and only the computer unit needs to be put into a “stand-by” mode, which permits said computer unit to be put into an operating state, in which reconstructions with already available projection data records are carried out, by way of a trigger signal from the networked system.

In accordance with the CT system according to at least one embodiment of the invention, the inventors also propose a method for reconstructing and diagnosing visual CT renderings, wherein at least one CT system comprising a computer unit with a memory and at least one diagnosis station spatially separate from the CT system and comprising a diagnosis computer with a memory are connected to one another via a communications network, wherein program code for carrying out the method steps described below is stored in the memories of the computer unit and of the diagnosis computer. Accordingly, the following is intended:

a request for reconstructing a CT data record with a projection data record stored in the computer unit is to be sent from a diagnosis computer in a diagnosis station to the computer unit of the CT system via the communications network,

the computer unit is to recognize this request,

the computer unit is to automatically execute the requested reconstruction, that is to say, with no additional action by operating staff, and

the reconstructed CT rendering is to be transmitted to the requesting diagnosis computer via the communications network.

Advantageously, the diagnosis computer can send at least one reconstruction parameter in the process, wherein the at least one reconstruction parameter is used in the reconstruction by the computer unit and reconstruction parameters not transmitted with the request are replaced by standard prescriptions.

Furthermore, the diagnosis computer can transmit a unique patient identification with the request, by which patient identification a projection data record, which was previously generated with the CT system and which is stored in the CT system, is determined.

Since some patients experience multiple CT scans, for example for different purposes as well, if a plurality of projection data records are available, a selection can also be provided for the diagnosis computer for a unique patient identification on the computer unit, and the projection data record to be used for the reconstruction can be determined by the diagnosis computer. Alternatively, if it is known that a plurality of projection data records from various examinations are present, it is also possible to define in the request for the reconstruction which projection data record is to be used for the reconstruction.

Moreover, according to at least one embodiment of the invention, a request profile, which defines a diagnostic object, can also be sent from the diagnosis computer to the computer unit and the computer unit extracts the reconstruction parameters from a predetermined table, in which reconstruction parameters which have an ideal configuration for the diagnostic question are stored for each request profile, on the basis of the request profile.

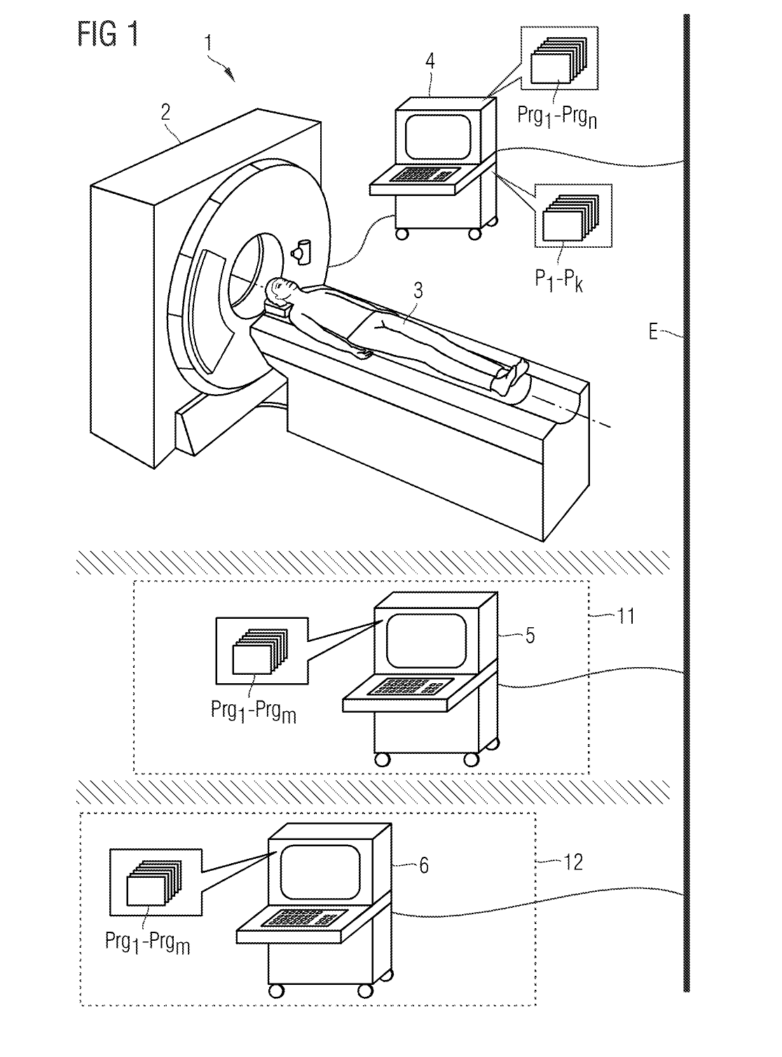

FIG. 1 shows the networked system according to an embodiment of the invention, comprising a CT system 1 and two diagnosis stations 11 and 12, which can interchange data amongst themselves via a communications network E, in this case an Ethernet connection, and, by way of network addresses, can respond individually amongst themselves. The CT system 1 comprises a gantry 2 with an emitter/detector system which, during operation, can scan a patient 3 and, in the process, generate at least one projection data record P1-Pk. Here, the projection data records can differ from one another in multifaceted ways as a result of very different scanning conditions. By way of example, a first projection data record can be generated with a first x-ray energy spectrum and a second projection data record can be generated with a second x-ray energy spectrum. A different projection data record can, for example, be generated with EKG-gating for a cardiac examination or it is possible to use different contrast agents during the examination in order to determine the wash-in in specific tissues, organs and blood vessels.

Such projection data records, which then form the base data for a reconstruction of CT renderings, are stored in a memory of a computer unit 4 and can be reconstructed with different diagnostic goals using different reconstruction parameters, for example in respect of the used slice thickness, the used HU-value window or in respect of the kernel used for the reconstruction.

If it becomes clear during the diagnosis at one of the diagnosis stations 11 or 12 that, in addition to the CT rendering which was generated under specific boundary conditions and which is available in any case, it would be more expedient to use a new CT rendering, which was scanned and reconstructed under different diagnostically meaningful conditions, the diagnosis computer 5 or 6, which is likewise connected to the communications network E, is able to direct a trigger signal via the communications network E for a query directly to the computer unit 4 of the CT system 1 and a new reconstruction can be triggered on the basis of already available projection data. The direct query of the diagnosis computer 5 or 6 to the computer unit 4 can take place in the case of an Ethernet connection between the computers 4, 5 and 6, for example by using a direct IP address. However, a name which is unique in the respective networked system can also be used for addressing, which name is resolved into an IP address in a manner known per se by way of a name server likewise present in the networked system.

By way of example, suspicions in respect of a lung diagnosis may arise when carrying out the diagnosis of a CT rendering substantially directed to an ideal bone rendering. However, a verified assessment is not possible with the present CT rendering. Thus, it is now possible via the communications network E to trigger an appropriate new reconstruction, in which a reconstruction kernel ideal for the lung diagnosis, e.g. B606, with a slice thickness of 1 mm is used.

It is also possible, for an improved diagnosis, to commission a new reconstruction of older available projections, the reconstruction parameters of which now exactly correspond to the reconstruction parameters of a current CT representation in order to obtain an ideal comparison in respect of the development of a tumor in the case of an oncological diagnosis or in respect of the development of calcification in coronary arteries.

As a result of the embodiment, according to the invention, of the CT system 1, more precisely of the programs Prg1-Prgn present in the computer unit 4, it is now no longer necessary for the CT system 1 to be occupied by operating staff. This is because the programming according to the invention now permits external triggering of reconstructions under predetermined conditions and with predetermined projection data records. For the run-through of such reconstruction, it is not necessary here for the complete CT system 1 to be put into operation; it suffices for the stand-by state of the computer unit 4 of the CT system 1 to be lifted and for only this computer unit 4 to become active in order to communicate with a diagnosis computer 5 or 6, with stored program code Prg1-Prgm of a diagnosis station 11 or 12 situated at a different locality, in order to obtain specific reconstruction parameters or a specific request profile. Under the predetermined conditions, the reconstruction can then be carried out on the computer unit 4 and the result can be transmitted to the diagnosis computer 5 or 6.

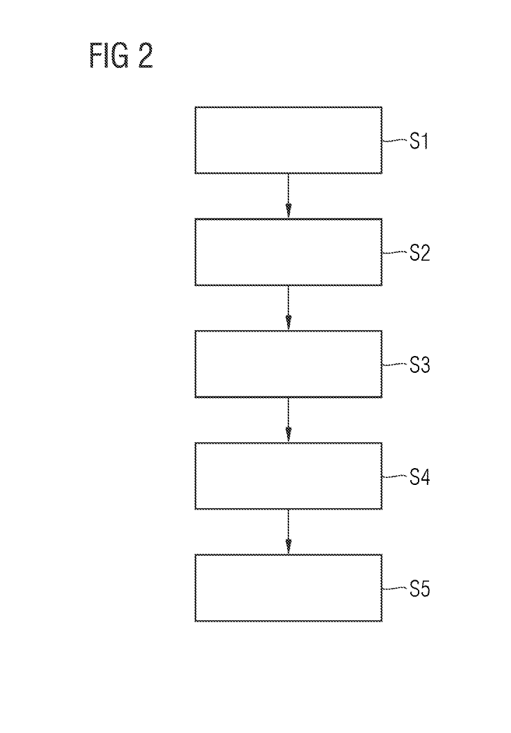

The method process of such a workflow can progress as depicted in an example manner in FIG. 2. In step S1, the diagnosis is started at a diagnosis station, wherein at least one CT rendering is available for this. Within the scope of this diagnosis, it turns out in step S2 that it would be expedient to have available a further CT rendering, which was reconstructed with reconstruction parameters adapted to the specific diagnostic question now posed.

Thereupon, a reconstruction request is directed in step S3 to the computer unit of the CT system via the communications network, which request, if necessary, first puts the computer unit into the operating mode from the stand-by mode and communicates the desired reconstruction boundary conditions, including a patient identification. This may be the specification of specific or all reconstruction parameters. However, it may also specify what diagnostic question is present such that the first computer unit, on the basis of an available table, uses the reconstruction parameters which are ideal for the diagnostic question, in conjunction with the projection data ideal for this.

In step S4, the desired reconstruction is carried out in the computer unit of the CT system and, in step S5, it is transmitted to a diagnosis computer of a diagnosis station.

Thus, overall, an embodiment of the invention presents a CT system comprising a computer unit and at least one memory device for storing program code and projection data records, which were established during a CT scan and which can be used for reconstructing CT renderings, wherein the computer unit is connected in a networked system with at least one external diagnosis computer comprising a memory with program code for diagnosing reconstructed visual CT renderings, and also a method for reconstructing and diagnosing visual CT renderings. Here, according to an embodiment of the invention, a request for reconstructing a CT data record with a projection data record stored in the computer unit is sent from a diagnosis computer in a diagnosis station to the computer unit of the CT system via a communications network, wherein the computer unit recognizes this request, the computer unit automatically executes the requested reconstruction and the reconstructed CT rendering is transmitted to the requesting diagnosis computer via the communications network.

Although the invention was illustrated and described more closely in detail by the preferred example embodiment, the invention is not restricted by the disclosed examples and other variations can be derived therefrom by a person skilled in the art, without departing from the scope of protection of the invention.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A CT system comprising:
a computer unit; and
at least one memory device configured to store first program code, executable during the operation, and to store projection data records, established during a CT scan and useable for reconstructing CT renderings, the computer unit being connected in a networked system with at least one external diagnosis computer comprising a memory configured to store second program code for diagnosing reconstructed visual CT renderings, the first program code being executable both during operation and in a “stand-by” state, wherein a new reconstruction of a CT rendering from a projection data record previously stored in the memory is executable or taskable, controlled by the first program code and an external trigger signal emitted by a diagnosis computer present in the networked system, and wherein the newly reconstructed CT rendering is sendable to the diagnosis computer.

2. The CT system of claim 1, wherein at least one of at least one CT slice image and a 3D rendering is reconstructed as the newly reconstructed CT rendering.

3. The CT system of claim 1, wherein the first program code available in the memory of the computer unit is configured to bring about the reception of at least one parameter which permits a unique identification of the projection data record to be used for the reconstruction.

4. The CT system of claim 1, wherein the first program code available in the memory of the computer unit is configured to bring about the reception of at least one reconstruction parameter, used for the reconstruction.

5. The CT system of claim 1, wherein the first program code available in the memory of the computer unit is configured to automatically replace non-received reconstruction parameters with standard values.

6. The CT system of claim 1, wherein, after receiving a trigger signal, the first program code available in the memory of the computer unit is configured to put the computer unit of the CT system out of the “stand-by” state and into an operating state without activating the gantry, x-ray tubes and detector.

7. A method for reconstructing and diagnosing visual CT renderings, at least one CT system comprising a computer unit including a memory, at least one diagnosis station spatially separate from the CT system and a diagnosis computer including a memory being connected to one another via a communications network, and program code configured to carry out the method described below being stored in the memories of the computer unit and of the diagnosis computer, the method comprising:
receiving a request for reconstructing a CT data record, with a projection data record stored in the computer unit, at the computer unit of the CT system, sent from the diagnosis computer in a diagnosis station via the communications network;
recognizing, at the computer unit, the received request for reconstruction;
automatically executing, at the computer unit, the requested reconstruction to produce a reconstructed CT rendering; and
transmitting the reconstructed CT rendering to the requesting diagnosis computer via the communications network.

8. The method of claim 7, wherein the computer unit of the CT system receives, from the diagnosis computer, at least one reconstruction parameter, wherein the at least one reconstruction parameter is used in the reconstruction by the computer unit.

9. The method of claim 7, wherein reconstruction parameters received from the diagnosis computer with the request, are replaced by standard prescriptions.

10. The method of claim 7, wherein a unique patient identification, sent from the diagnosis computer, is received with the request, by which patient identification the associated projection data record is determined.

11. The method of claim 7, wherein, if a plurality of projection data records are available, a selection is provided for the diagnosis computer for a unique patient identification on the computer unit, and the projection data record to be used for the reconstruction is determined by the diagnosis computer.

12. The method of claim 7, wherein a request profile is received, from the diagnosis computer, at the computer unit and the computer unit extracts the reconstruction parameters from a table, the reconstruction parameters being stored for each request profile, on the basis of the request profile.

13. The CT system of claim 2, wherein the first program code available in the memory of the computer unit is configured to bring about the reception of at least one parameter which permits a unique identification of the projection data record to be used for the reconstruction.

14. The CT system of claim 2, wherein the first program code available in the memory of the computer unit is configured to bring about the reception of at least one reconstruction parameter, used for the reconstruction.

15. The CT system of claim 2, wherein the first program code available in the memory of the computer unit is configured to automatically replace non-received reconstruction parameters with standard values.

16. The CT system of claim 2, wherein, after receiving a trigger signal, the first program code available in the memory of the computer unit is configured to put the computer unit of the CT system out of the “stand-by” state and into an operating state without activating the gantry, x-ray tubes and detector.

17. The method of claim 8, wherein reconstruction parameters received from the diagnosis computer with the request, are replaced by standard prescriptions.

18. The method of claim 8, wherein a unique patient identification, sent from the diagnosis computer, is received with the request, by which patient identification the associated projection data record is determined.

19. The method of claim 8, wherein, if a plurality of projection data records are available, a selection is provided for the diagnosis computer for a unique patient identification on the computer unit, and the projection data record to be used for the reconstruction is determined by the diagnosis computer.

20. The method of claim 8, wherein a request profile is received, from the diagnosis computer, at the computer unit and the computer unit extracts the reconstruction parameters from a table, the reconstruction parameters being stored for each request profile, on the basis of the request profile.
